(19) United States
(12) Patent Application Publication
(10) Pub. No.: US 2018/0309066 A1
(43) Pub. Date: Oct. 25, 2018

(54) Spirofluorene Compound and Luminescent Device Using Same

(71) Applicant: AAC Technologies Pte, Ltd., Singapore City (SG)

(72) Inventor: Zaifeng Xie, Shenzhen (CN)

(21) Appl. No.: 15/955,859

(22) Filed: Apr. 18, 2018

(30) Foreign Application Priority Data
Apr. 21, 2017 (CN) 201710265001.2

(51) Int. Cl. H01L 51/00; C07C 317/14; C09K 11/06; C07D 213/71

(52) U.S. Cl. CPC H01L 51/0056; C07C 317/14; C09K 11/06; C07D 213/71; H01L 51/0067; H01L 51/5012; C09K 2211/1014; C09K 2211/1011; C09K 2211/1007; C09K 2211/1018; C07C 2603/18

(57) ABSTRACT

The invention relates to the technical field of organic luminescent materials, in particular to a spirofluorene compound and a luminescent device thereof. Spiro difluorene compounds selected freely I compounds: Y₁ and Y₂ denote hydrogen, electron-absorbing groups or electron-donating groups independently, respectively; at least a substituent in X1 and X2 is the substituent shown in formula II; M denotes —S—, —P—, —SO—, —SO₂—, —S(═S)—, —S(═S)(═S)—, —PO—, —PO₂—, —P(═S)—, —P(═S)(═S)—, —C(═O)—; N₁, N₂, N₃ and N₄ denote carbon or nitrogen atoms independently, respectively; N is an integer of 0˜4. The spirobifluorene compound of the invention has A-D-A chemical structure, and a spatial dihedral angle of nearly 90° is formed between an electron D unit and an electron-absorbing A unit, which is good for HOMO-LUMO orbital separation of thermal activation of delayed fluorescence materials, in order to obtain ideal ΔEST.

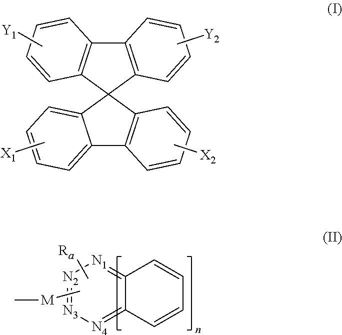

### FIELD OF THE PRESENT DISCLOSURE

The invention relates to a technical field of organic luminescent materials, in particular to a spirofluorene compound and a luminescent device thereof.

### DESCRIPTION OF RELATED ART

According to the electroluminescent mechanism, OLED materials can be divided into fluorescent OLED materials and phosphorescent OLED materials. The existing OLED technology materials, phosphor luminescent materials, due to the effect of heavy metals, theoretically can achieve a quantum luminous efficiency of 100%, especially a great progress have been made in red and green phosphor materials. However, the triplet excitons of phosphorescent materials are easy to be quenched at high concentration, so it is necessary to maintain a certain proportion of host and guest doping in order to improve the properties of phosphorescent materials.

Fluorescent OLED material is a type of pure organic material, which contains no heavy metals. Therefore, theoretically, it can only reach the internal quantum efficiency of 25%, resulting in 5% upper limit of the theoretical external quantum efficiency of fluorescence. At present, the red and green OLED materials have made great progress, and the properties of fluorescent blue OLED materials have not been comparable with other red and green materials.

Recently, the materials of thermally activated delayed fluorescence (TADF) have attracted much attention. Due to the orbital separation of HOMO-LUMO, the triplet excitons can jump to the singlet orbits in a thermal way, thus obtaining an internal quantum efficiency of nearly 100%. However, blue TADF material is still a difficulty at present, because in order to obtain fluorescent blue with high color purity, the triplet state of organic materials is at least required to be above 2.6 ev and the high performance is maintained. Worldwide, many scientific research institutes are devoted to the development of this kind of materials, but the results are very few.

Therefore, this invention is proposed.

### BRIEF DESCRIPTION OF THE DRAWINGS

Many aspects of the exemplary embodiments can be better understood with reference to the following drawings. The components in the drawing are not necessarily drawn to scale, the emphasis instead being placed upon clearly illustrating the principles of the present disclosure.

FIG. 1 is a structural diagram of a luminous device of the invention;

FIG. 2 is a schematic diagram of A-D-A spatial configuration of a spirobifluorene compound in the invention;

FIG. 3 is another A-D-A spatial configuration diagram of the spirobifluorene compound in the invention;

FIG. 4 shows an UV absorption spectrum of a compound SDF-DPSO2;

FIG. 5 shows a NMR carbon spectrum of a compound SDF-DPSO2;

FIG. 6 shows a nuclear magnetic resonance hydrogen spectrum of a compound SDF-DPSO2;

FIG. 7 shows a molecular ground state simulation diagram of a compound SDF-DPSO2;

FIG. 8 shows a NMR carbon spectrum of a compound SDF-DPYSO2;

FIG. 9 shows a nuclear magnetic resonance hydrogen spectrum of a compound SDF-DPYSO2;

FIG. 10 shows a NMR carbon spectrum of a compound SDF-DPSOcl2;

FIG. 11 shows a nuclear magnetic resonance hydrogen spectrum of a compound SDF-DPSOcl2;

FIG. 12 shows a NMR carbon spectrum of a compound SDF-4PySOcl;

FIG. 13 shows a nuclear magnetic resonance hydrogen spectrum of a compound SDF-4PySOcl.

10—luminescent device;
11—anode;
12—hole transport layer;
13—luminescent layer;
14—electronic transport layer;
15—cathode.

### DETAILED DESCRIPTION OF THE EXEMPLARY EMBODIMENTS

The present disclosure will hereinafter be described in detail with reference to several exemplary embodiments. To make the technical problems to be solved, technical solutions and beneficial effects of the present disclosure more apparent, the present disclosure is described in further detail together with the figure and the embodiments. It should be understood the specific embodiments described hereby is only to explain the disclosure, not intended to limit the disclosure.

The present invention is further elaborated in combination with exemplary embodiments. It should be understood that these embodiments are used only to illustrate the invention and not to limit the scope in the invention.

The invention relates to a spirobifluorene compound, and the spirobifluorene compound is selected from the general formula I:

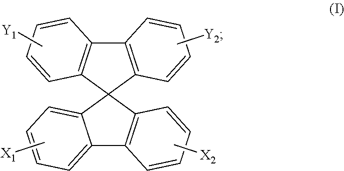

Among them, Y₁, Y₂ denote hydrogen, electron absorption group or electron-donating group independently, respectively;

At least a substituent in X₁, X₂ is the substituent shown in Formula II:

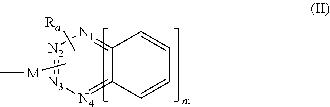

M denotes —S—, —P—, —SO—, —SO₂—, —S(═S)—, —S(═S)(═S)—, —PO—, —PO₂—, —P(═S)—, —P(═S)(═S)—, —C(═O)—;

N₁, N₂, N₃, N₄ denote carbon or nitrogen atoms independently, respectively;

Rₐ is selected from hydrogen, halogen, C_{1˜30} alkyl, C_{1˜30} alkyl substituted by hydroxyl or C_{6˜48} alkylaryl;

n is an integer of 0˜4.

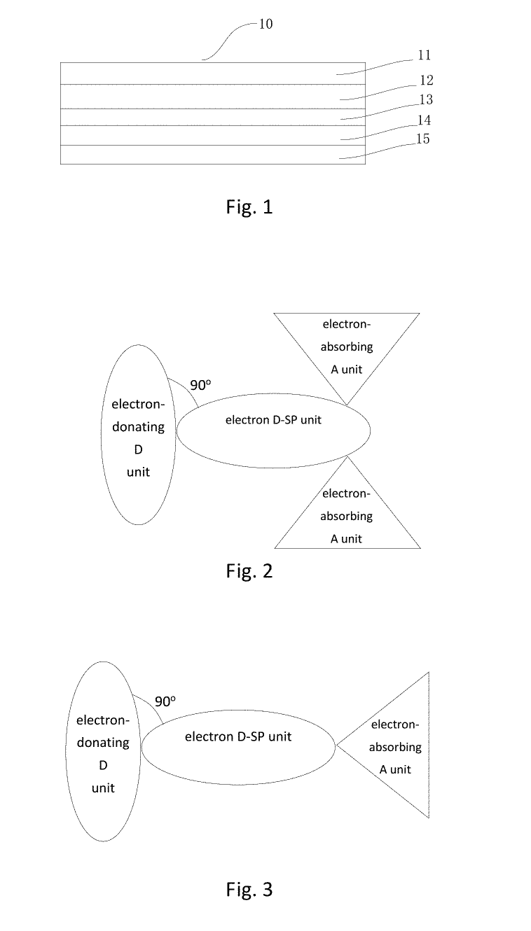

The spirobifluorene compound proposed by the invention has an A-D-A chemical structure and a schematic diagram of its spatial configuration as shown in FIGS. 1 and 2. As shown in FIGS. 1 and 2, a spatial dihedral angle of nearly 90° is formed between an electron-donating D unit and an electron-absorbing A unit, which is good for HOMO-LUMO orbital separation of the thermal activation delayed fluorescence material. The spirobifluorene compound of the present invention obtains pure blue light spectrum by holding high triplet and singlet energy level of the electron D-SP unit, and a nearly 90° spatial structure is formed between the electron-donating D unit and the electron-absorbing A unit in the invention, which is good for preventing the spectrum redshift caused by the conjugation between the electron-donating D and SP and between the electron-donating D and the electron-absorbing A.

As an improvement of the spirobifluorene compound of the present invention, the spirobifluorene compound is selected from the compounds shown in the general formula IA:

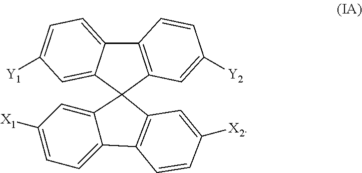

The substitution sites of X₁, X₂, Y₁, Y₂ in formula IA are the 2,7 substituents of fluorene, which are the most chemically active sites of fluorene, so the compounds expressed in formula IA are easier to synthesize.

As an improvement of the spirobifluorene compound of the invention, when Y₁, Y₂ are an electron-donating group and an electron-donating group (known as electron-giving groups), Y₁, Y₂ are selected from substituted or unsubstituted C_{1˜30} alkyl groups, substituted or unsubstituted diphenyl groups, or substituents expressed by the following structural formulas independently, respectively:

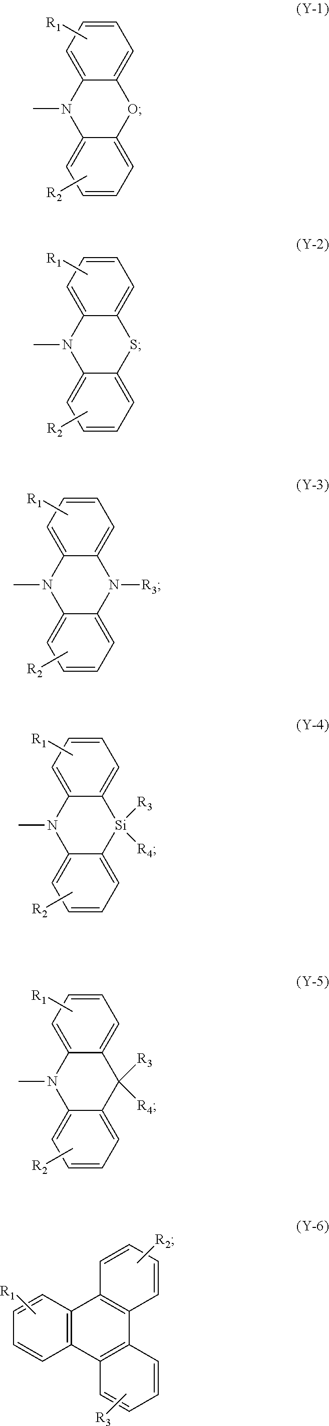

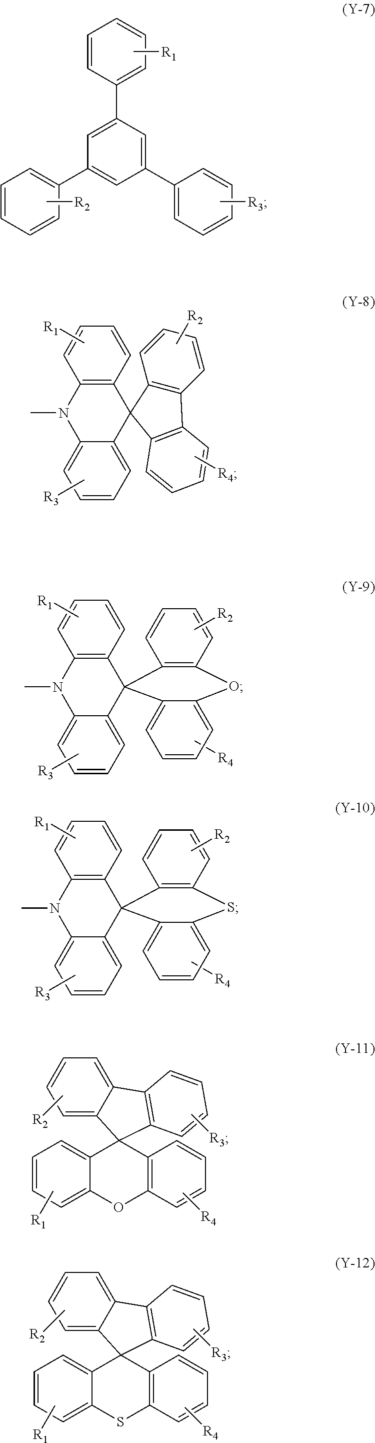

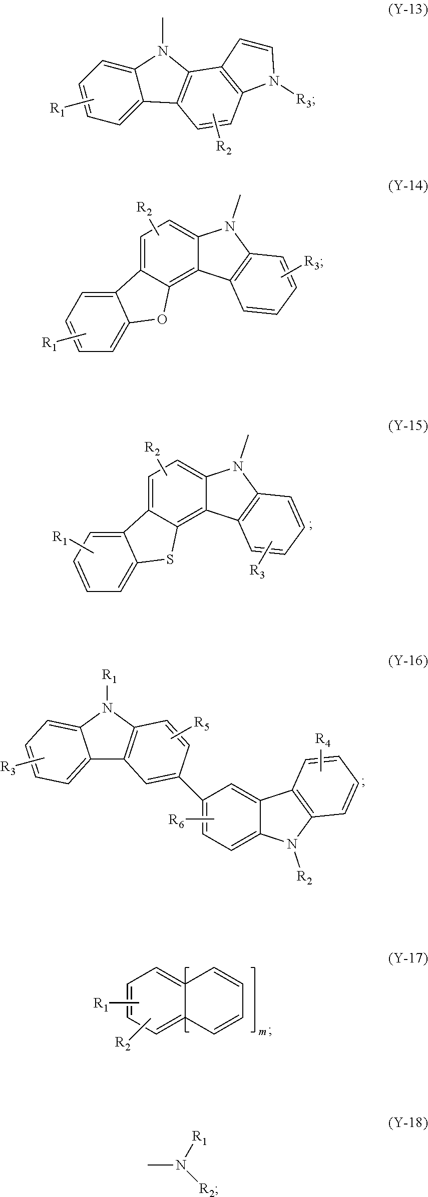

Among them, R₁, R₂, R₃, R₄ are selected from hydrogen atoms, amino groups, halogens, substituted or unsubstituted C_{1˜12} alkyl, substituted or unsubstituted C_{1˜12} alkoxyl, substituted or unsubstituted C_{6˜12} aryl groups, substituted or unsubstituted C_{6˜12} aryloxy groups;

A substituent is selected from halogen atoms, alkyl groups of C_{1˜12}, alkyl groups of C_{1˜12} substituted by halogen atoms and alkoxyl groups of C_{1˜12} substituted by halogen atoms.

m is an integer of 0˜4;

Any hydrogen atom in benzene ring can be substituted by the formula Y-6, the formula Y-7, Y the formula-11, the formula Y-12, the formula Y-16 and the formula Y-17 to form a substituent.

As an improvement of the spirobifluorene compound in the invention, when Y₁, Y₂ are electron-absorbing group and an electron-donating group (known as electron-pulling groups), Y₁, Y₂ denote substituents shown in formula II independently, respectively.

As an improvement of the spirobifluorene compound in the present invention, in formula I, X₁ is selected from hydrogen or a substituent shown in the formula IIa, and X₂ is selected from a substituent shown by the formula IIa;

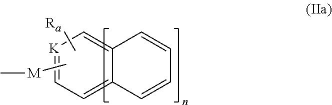

K is a carbon or nitrogen atom.

As an improvement of the spirobifluorene compound in the invention, in the formula IIa, M denotes —SO—, —SO₂—, —PO—.

As an improvement of a spirobifluorene compound in the present invention, when Y₁, Y₂ are the electron-donating groups, the spirobifluorene compound can be selected from the compound shown by the general formula IA-1.

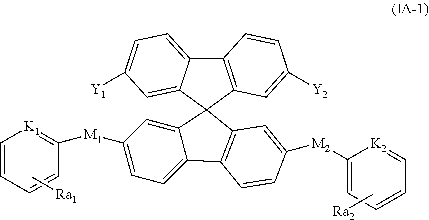

In IA-1, M 1, M 2 denote —SO—, —SO₂—, —PO-independently, respectively; Rₐ₁, Rₐ₂ are selected from hydrogen, halogen, C_{1˜12} alkyl, C_{6˜24} aryl groups independently, respectively; K₁, K₂ denote carbon or nitrogen atoms independently, respectively.

Y₁, Y₂ are selected from the electron-donating groups in the invention independently, respectively.

Further preferably, Rₐ₁, Rₐ₂ are selected from hydrogen atoms or halogens independently, respectively.

More preferably, M₁, M₂ are the same; Rₐ₁, Rₐ₂ are the same; K₁, K₂ are the same.

If Y₁, Y₂ are electron-donating substituents, i.e., the electron-donating substituents are added to the delocalized position of HOMO orbit, when the distribution of the HOMO orbit will be more scattered to these electron substituents. But it does not affect LOMO, and the material still maintains TADF properties.

As an improvement of a spirobifluorene compound in the present invention, when Y₁, Y₂ are hydrogen atoms, the spirobifluorene compound is selected from the general formula IA-2:

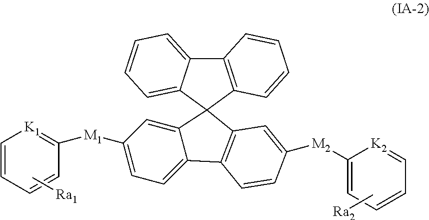

In IA-2, M₁, M₂ denote —SO—, —SO₂—, —PO-independently, respectively; Rₐ₁, Rₐ₂ are selected from hydrogen, halogen, C_{1˜12} alkyl, C_{6˜24} aryl groups; K₁, K₂ denote carbon or nitrogen atoms independently, respectively.

The further selected Rₐ₁ and Rₐ₂ are selected from hydrogen atoms or halogens.

The better selection of M₁, M₂ is the same as Rₐ₁, Rₐ₂ is the same as K₁, K₂ is the same.

As an improvement of the spirobifluorene compound of the present invention, when Y₁ and Y₂ are the electron-absorbing groups, the spirobifluorene compound is selected as the compound shown by the general formula IA-3.

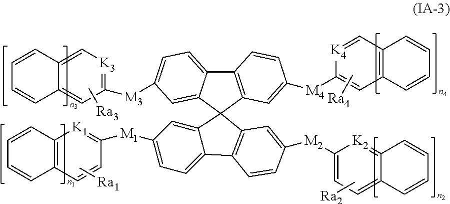

In IA-3, M₁, M₂, M₃ and M₄ denote —SO—, —SO₂—, —PO-independently. Rₐ₁, Rₐ₂, Rₐ₃ and Rₐ₄ are selected from hydrogen, halogen, C_{1˜12} alkyl group and C_{6˜24} aryl group; K₁, K₂, K₃, K₄ denote carbon or nitrogen atoms independently, respectively;

n₁, n₂, n₃, n₄ are selected from any integer of 0˜4 independently, respectively.

Further preferably, Rₐ₁, Rₐ₂, Rₐ₃, Rₐ₄ are selected from hydrogen atoms or halogens independently, respectively.

More preferably, M₁, M₂, M₃, M₄ are the same; Rₐ₁, Rₐ₂, Rₐ₃, Rₐ₄ are the same; K₁, K₂, K₃, K₄ are the same.

If Y₁, Y₂ are electron-absorbing substituents, the LUMO orbits are more delocalized and scattered, and there is less overlap with the HOMO orbits.

As an improvement of a spirobifluorene compound in the invention, when Y₁, Y₂ are hydrogen atoms, the spirobifluorene compound is selected from the compounds shown by the following structural formulas:

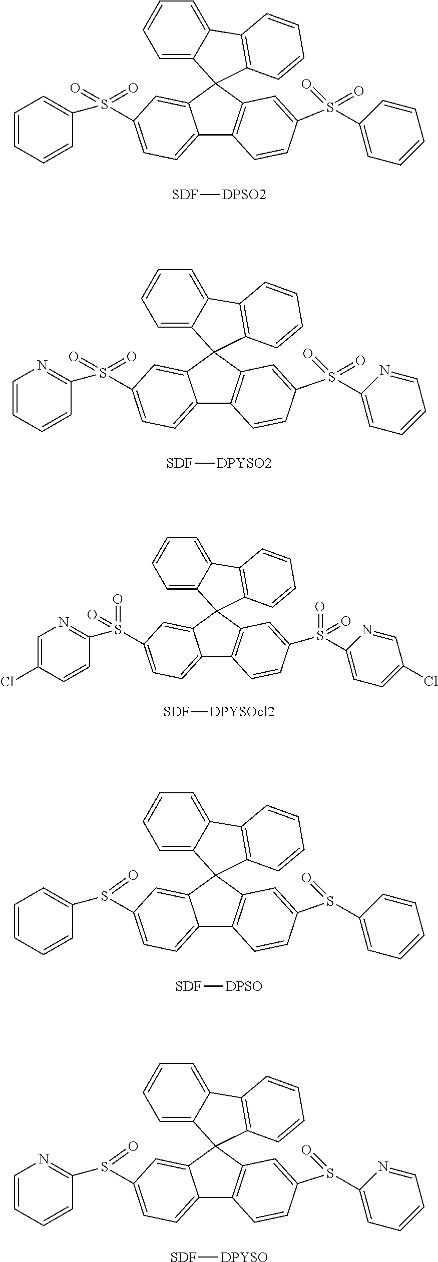

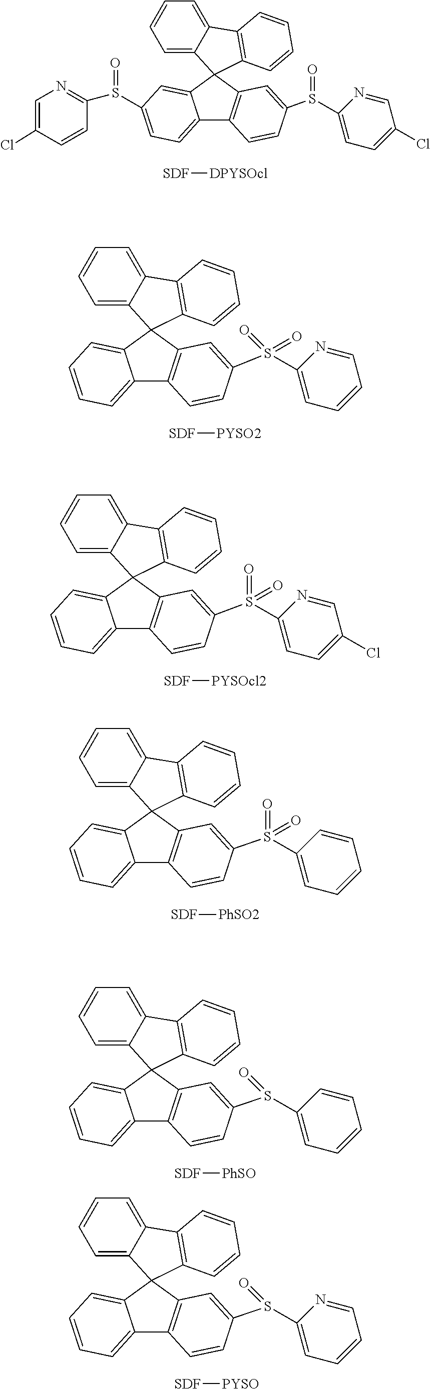

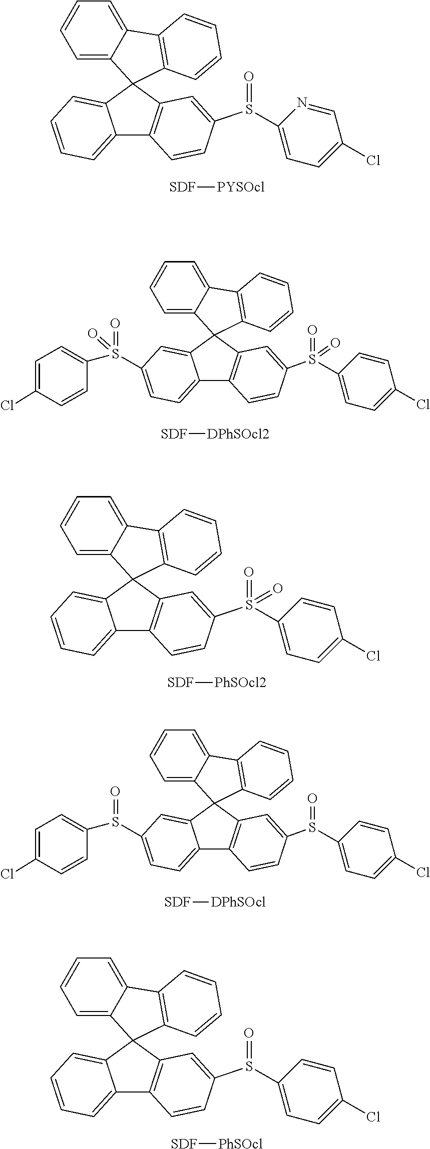

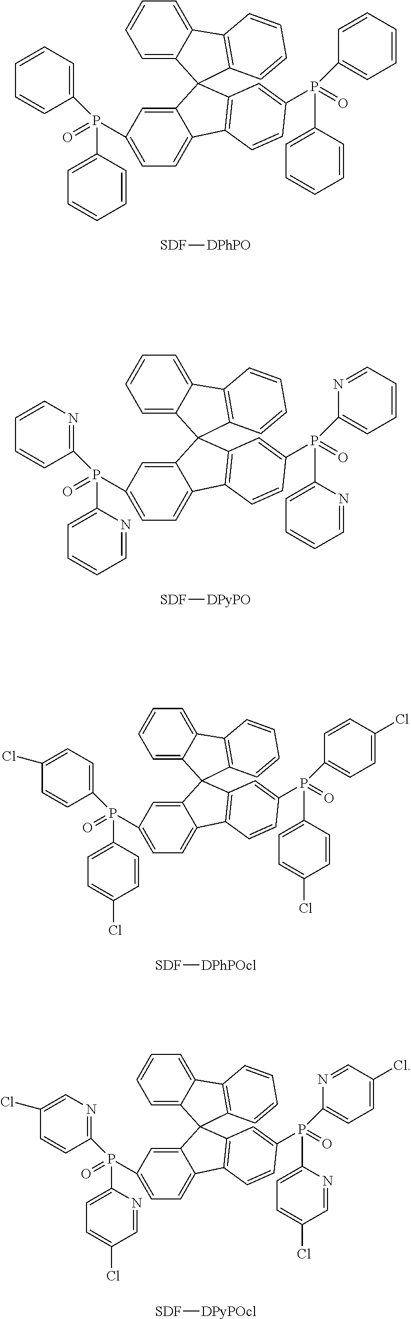

As an improvement of a spirobifluorene compound in the present invention, when Y₁, Y₂ are the electron-donating groups, the spirobifluorene compound is selected from the compounds shown by the following structure formulas:

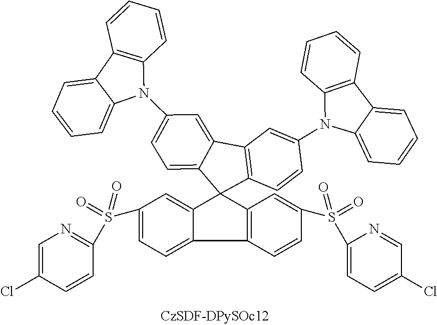 [SMILES: O=S(=O)(c1ccc2c(c1)C1(c3ccc(-n4c5ccccc5c5ccccc54)cc3-c3cc(-n4c5ccccc5c5ccccc54)ccc31)c1cc(S(=O)(=O)c3ccc(Cl)cn3)ccc1-2)c1ccc(Cl)cn1]

As an improvement of a spirobifluorene compound in the present invention, when Y₁, Y₂ are electron-absorbing groups, the spirobifluorene compound is selected from the compounds shown by the following structure formulas:

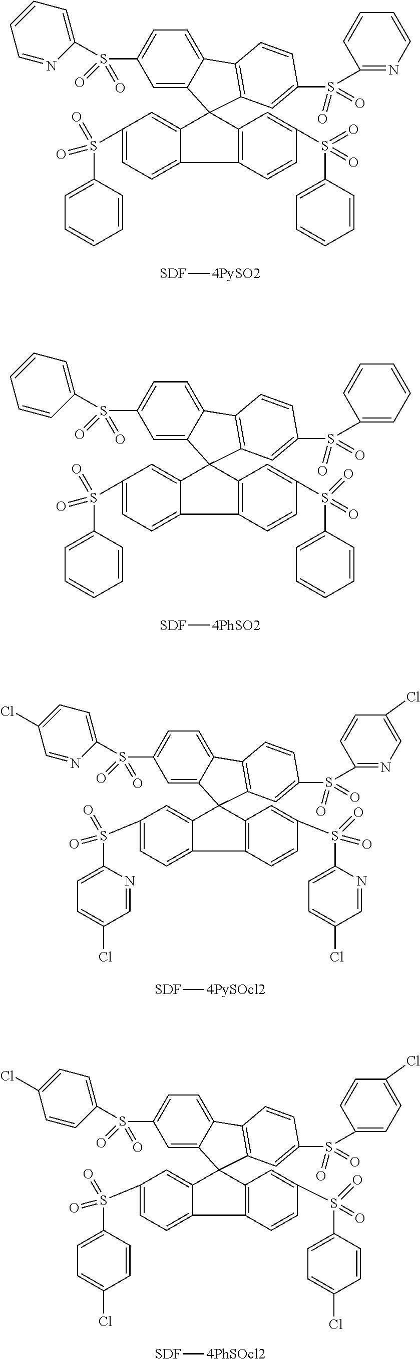

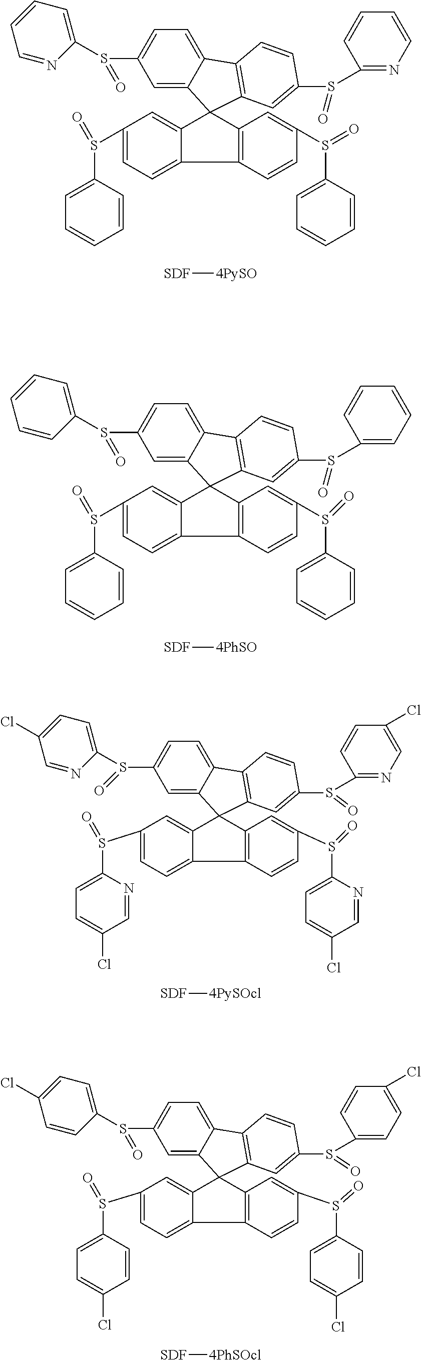

The synthetic route of a spirobifluorene is as follows:

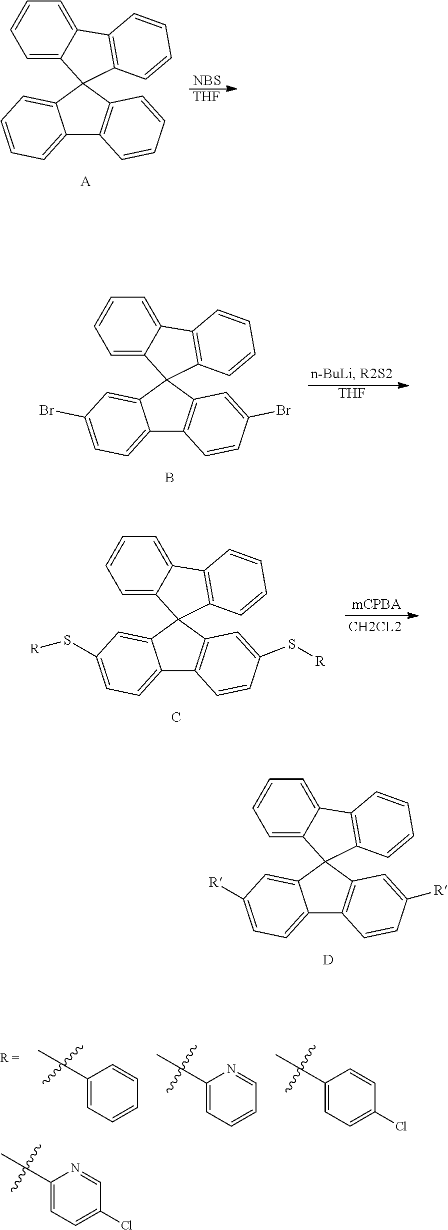

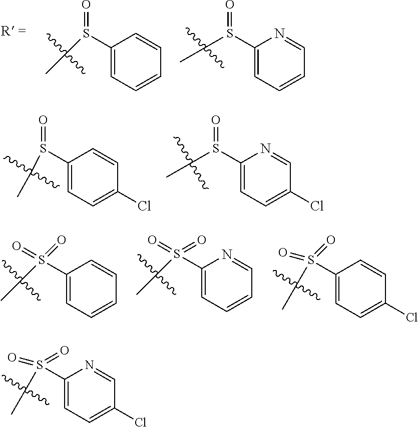

The specific process is as follows: the mixed solution of NBS(N-bromosuccinimide)/THF is added into the reaction bottle containing A, and heated to 40 [symbol] for 1 hour under the condition of nitrogen protection. B. Then, R—S—S—R, a disubstituted disulfide compound, is added, and n-BuLi/THF is used as a catalyst, which is stirred in a low temperature dry ice bath for half an hour to obtain compound C. Finally, the mixture of m-chlorobenzoic acid (mCPBA/CH₂Cl₂) solution is put into the mixed solution, and the mixture is stirred for 1 hour, then water is added to precipitate solid, then n-hexane is used to wash and ethanol is recrystallized to obtain D.

R can be benzene ring, pyridine, p-chlorobenzene, m-chloropyridine.

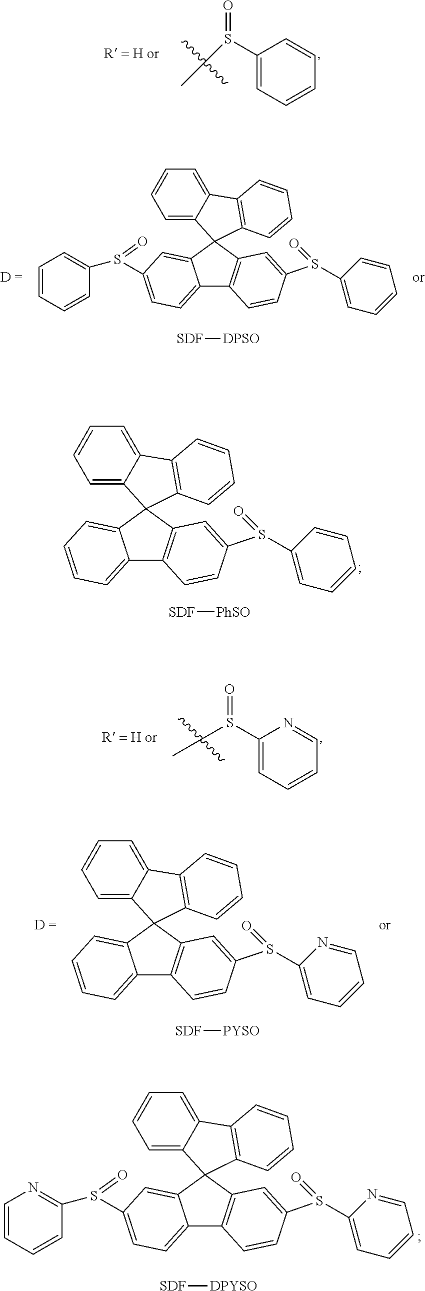

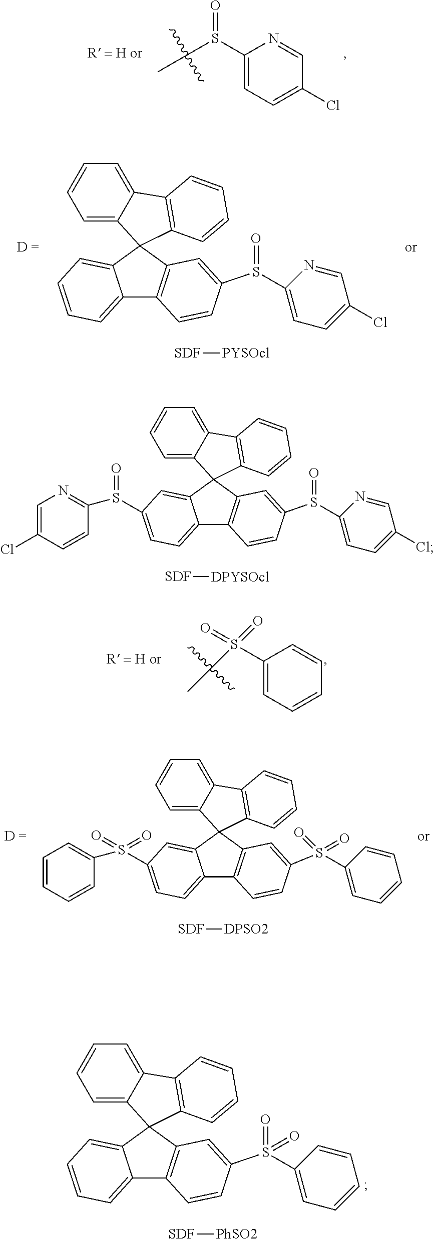

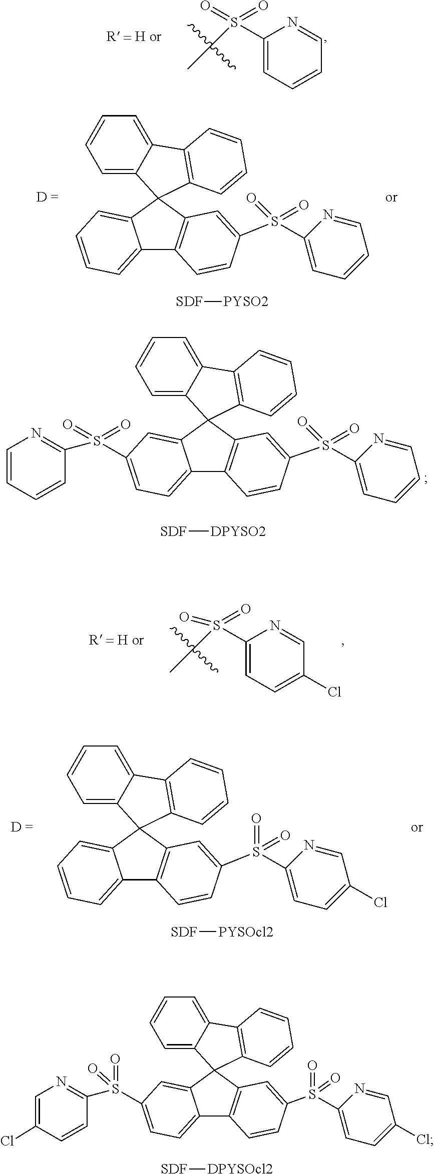

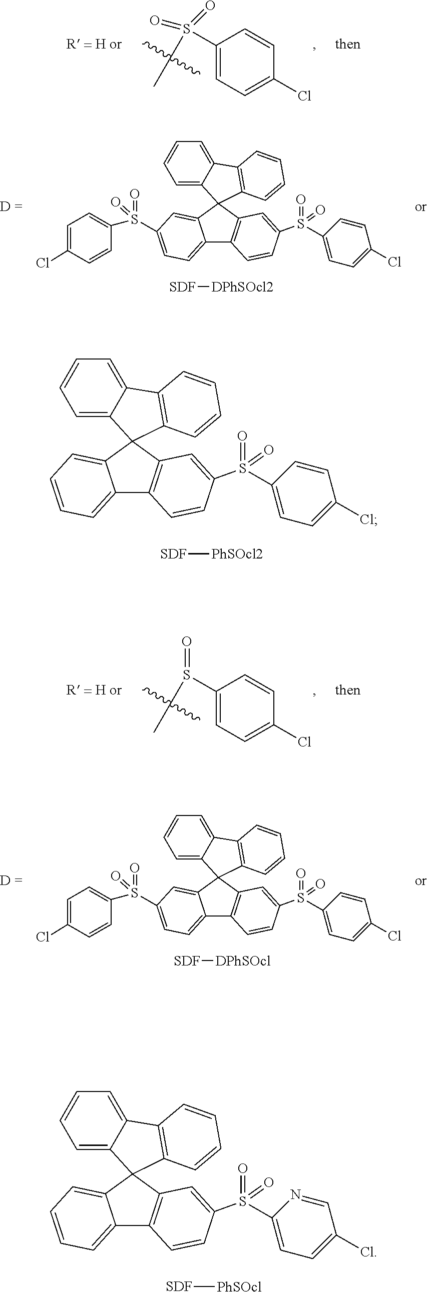

Further examples are given below to illustrate the synthesis of the materials of the present invention:

### Synthesis Embodiment 1: Synthesis of Compound SDF-DPSO2

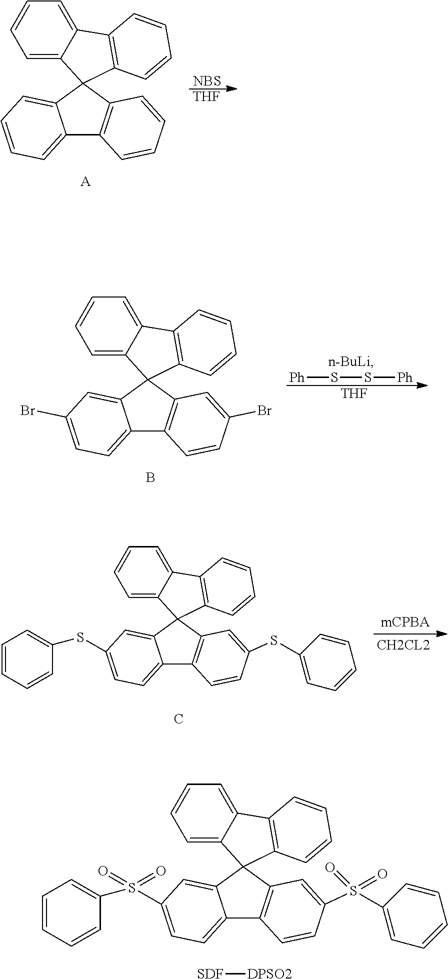

The mixed solution of NBS(N-bromosuccinimide)/THF is dripped into the reaction bottle containing 1 mol A and heated to 40 C for 1 hour under the condition of nitrogen protection, and the bromination reaction is carried out to obtain B. Then, diphenyl substituted disulfide compound Ph-S—S-Ph is added and n-BuLi/THF is added as a catalyst, which is stirred in low temperature dry ice bath for half an hour to obtain compound C. Finally, the mixed solution of m-chlorobenzoic acid (mCPBA/CH2Cl2) is put into the mixed solution, and then the mixture is stirred for 1 hour, water is added, the solid is precipitated, and then n-hexane is used to wash in turn and ethanol is recrystallized to obtain SDF-DPSO₂, and the yield is 38%.

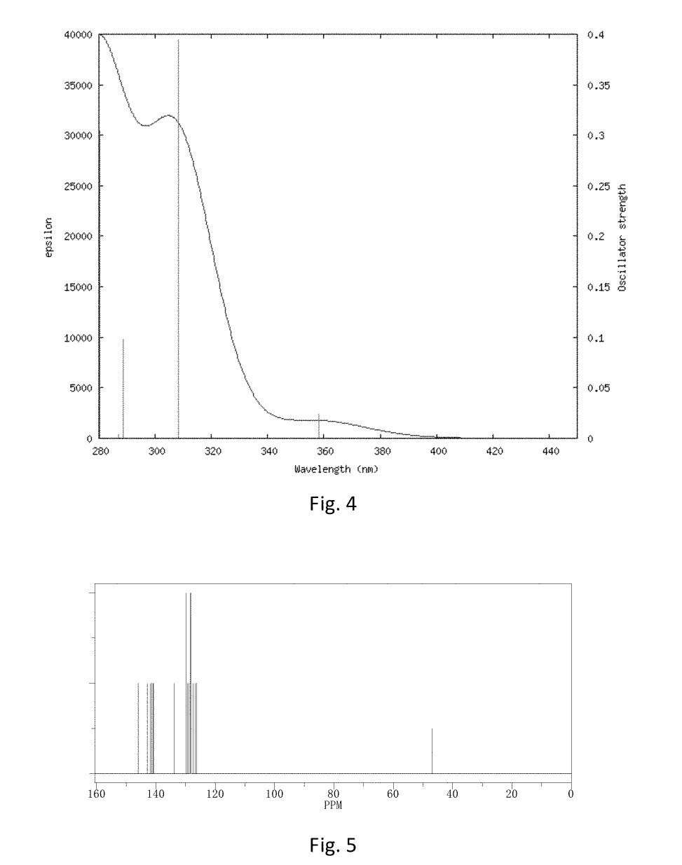

The UV absorption spectrum (CH2CL2) is shown in FIG. 4, according to the UV absorption spectrum, the compound has a strong absorption spectrum between 250 nm and 400 nm, of which the absorption intensity is the highest at 360 nm and 310 nm.

The main peak of photoluminescence spectrum (PL) is 435.22 nm, which is a kind of blue light material.

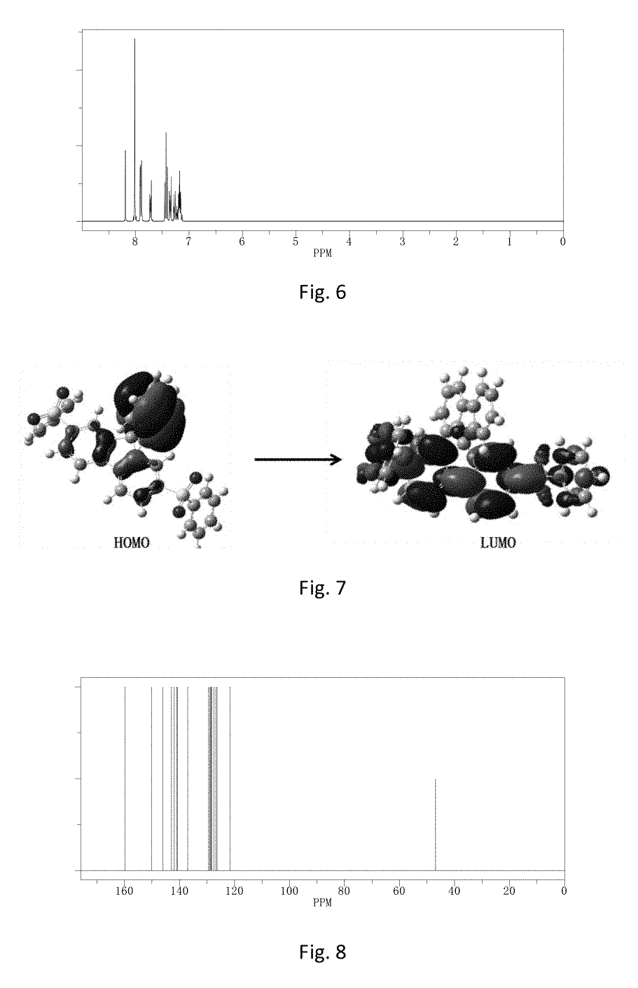

The NMR carbon spectrum is shown in FIG. 5, and the nuclear magnetic hydrogen spectrum is shown in FIG. 6.

The ground state structure of SDF-DPSO2 molecule is simulated by Gaussian 03 quantitative simulation software, and the molecular ground state simulation diagram is shown in FIG. 7 and the simulation diagram is shown in FIG. 7; It can be seen from FIG. 7 that the HOMO is distributed on the helical compounds, and LUMO is distributed on phenyl sulfoxide of the two sides, which is expected to reach the lower splitting energy of EST singlet state and triplet state. The HOMO-LUMO orbit is separated completely.

A time-dependent density functional method (TDDFT) is used to simulate the molecular configuration of the ground state of the material at B3LYP level. The bond length and bond angle are calculated, as shown in Table 1.

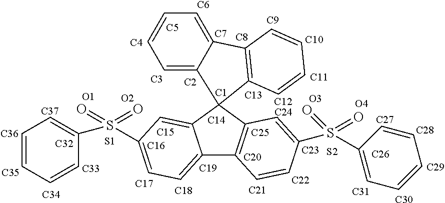

TABLE 1

| | Bond length A | Bond angle | Dihedral angle |
|---|---|---|---|
| C7-C8 | 1.46943 | | |
| C1-C2 | 1.53385 | | |
| C1-C13 | 1.53336 | | |
| C1-C14 | 1.53053 | | |
| C1-C25 | 1.53053 | | |
| S1-C16 | 1.80379 | | |
| S1-C32 | 1.80352 | | |
| S2-C23 | 1.80379 | | |
| S2-C26 | 1.80352 | | |
| C2-C1-C13 | | 101.425 | |
| C14-C1-C25 | | 101.086 | |
| C32-S1-C16 | | 104.771 | |
| C23-S2-C26 | | 104.770 | |
| C13-C1-C2-C14 | | | 122.506 |

According to the molecular data in Table 1, it can be seen that SDF-DPSO2 compounds maintain good molecular symmetry, with a dihedral angle of C13−C1−C2−C14=122°, in order to separate the HOMO-LUMO.

### Synthesis Embodiment 2: Synthesis of Compound SDF-DPYSO2

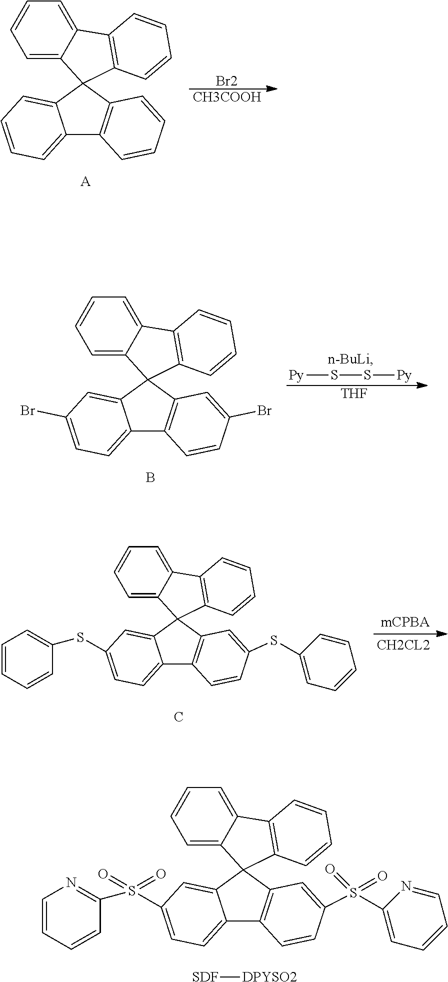

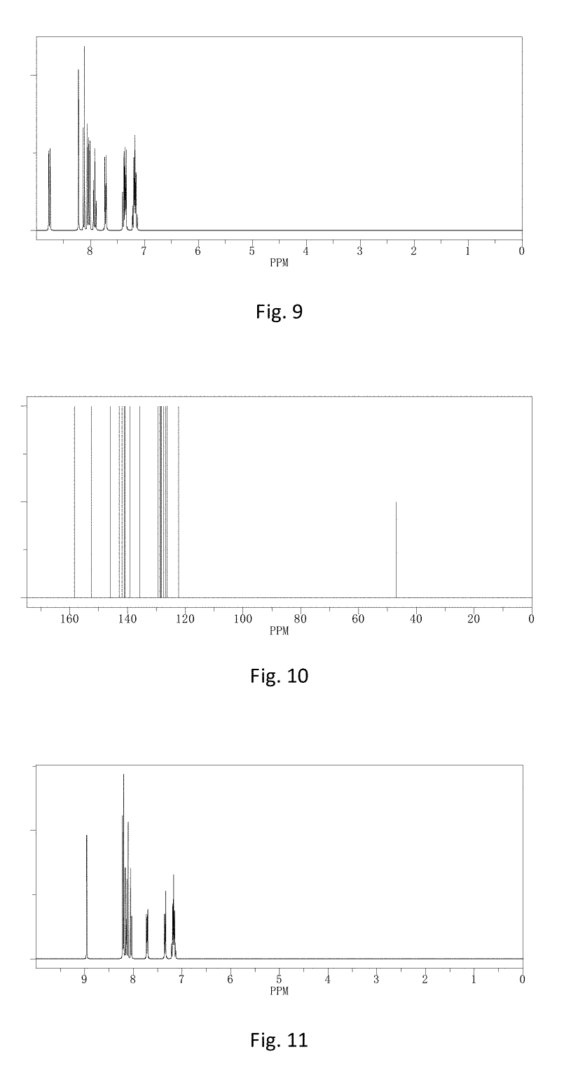

In the reaction bottle containing 1 mol A, the mixed solution with Br₂/CH₃COOH is dripped, and the bromination reaction is carried out under the condition of nitrogen protection to obtain B. Then, the disulfide compound Py-S—S-Py is added, and n-BuLi/THF is used as a catalyst, and the compound C is obtained by stirring in a low temperature dry ice bath for half an hour. Finally, the mixed solution of m-chlorobenzoic acid (mCPBA/CH₂Cl₂) is put into the mixed solution, and then the mixture is stirred for 1 hour, and the water is added, and the solid is precipitated, and then n-hexane is used to wash in turn and ethanol is recrystallized to obtain SDF-DPySO2 with the yield of 46%. The NMR carbon spectrum is shown in FIG. 8, and the nuclear magnetic hydrogen spectrum is shown in FIG. 9.

### Synthesis Embodiment 3: Synthesis of Compound SDF-DPYSOcl2

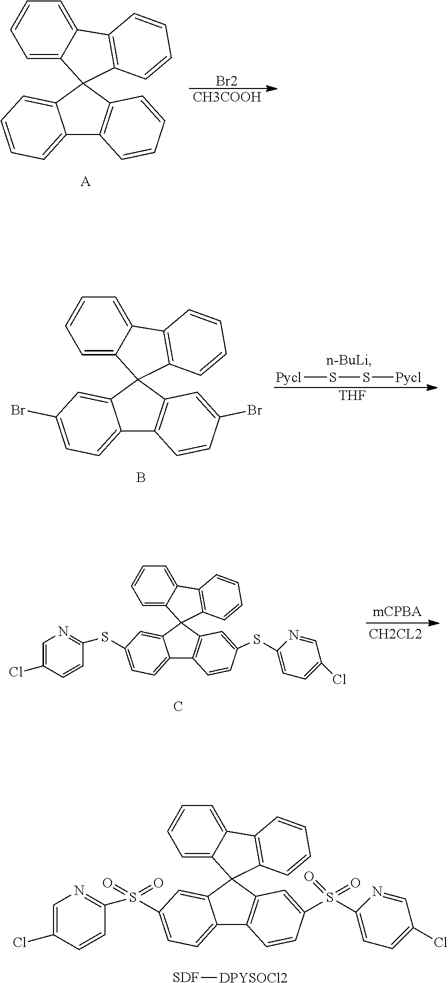

In the reaction bottle containing 1 mol A, the mixed solution with Br₂/CH₃COOH is dripped, and the bromination reaction is carried out under the condition of nitrogen protection to obtain B. Then, the bispyridine substituted disulfide compound Pycl-S—S-Pycl is added, and n-BuLi/THF is used as catalyst, and the compound C is obtained by stirring in a low temperature dry ice bath for half an hour. Finally, the mixed solution of m-chlorobenzoic acid (mCPBA/CH₂Cl₂) is put into the mixed solution, and then the mixture is stirred for 1 hour, water is added, the solid is precipitated, and then n-hexane is used to wash and ethanol is recrystallized to obtain SDF-DPySOcl2 with the yield of 43%. The nuclear magnetic resonance carbon spectrum is shown in FIG. 10 and the nuclear magnetic hydrogen spectrum is shown in FIG. 11.

### Synthesis Embodiment 4: Synthesis of Compound SDF-4Py SOcl

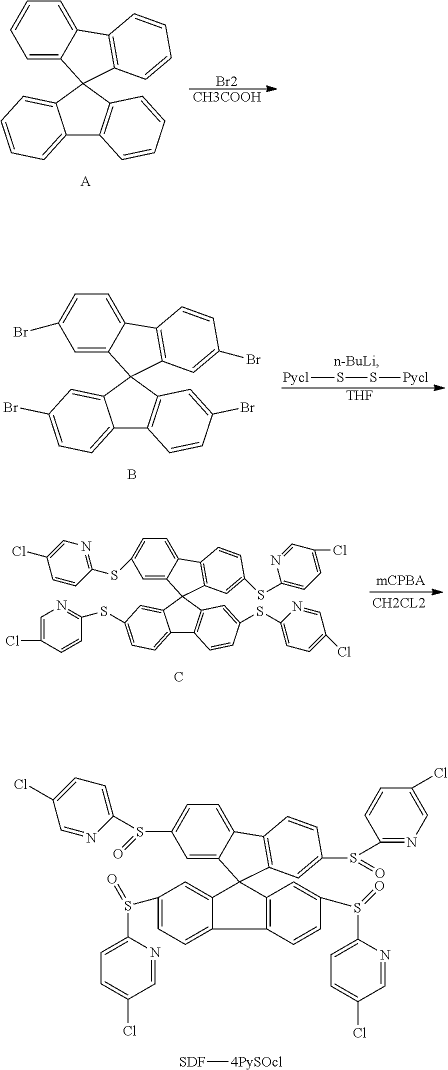

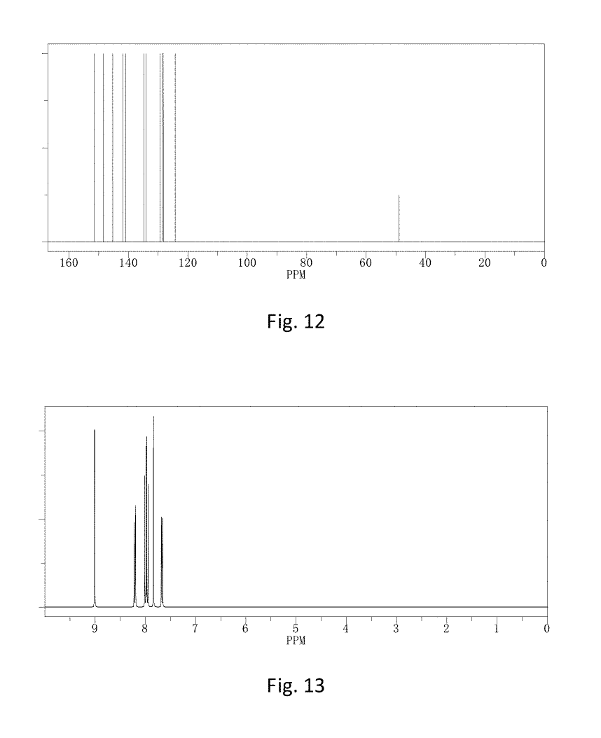

B is obtained by bromination reaction under the condition of nitrogen protection by dropping the mixed solution with Br₂/CH₃COOH in the reaction bottle containing A. Then, the dichloropyridine substituted disulfide compound Pycl-S—S-Pycl is added, and n-BuLi/THF is used as a catalyst, which is stirred in a low temperature dry ice bath for half an hour t obtain compound C. Finally, the mixed solution of m-chlorobenzoic acid mCPBA/CH₂Cl₂ is put into the mixed solution, and the mixture is stirred for 1 hour, and the water is added, and the solid is precipitated, then n-hexane is used to wash in turn and ethanol is recrystallized to obtain SDF-4PySOcl with the yield of 63%. The nuclear magnetic resonance carbon spectrum is shown in FIG. 12 and the nuclear magnetic hydrogen spectrum is shown in FIG. 13.

ΔEST Test

In general organic materials, S1 excited state and T1 excited state energy are different due to the different spins, and the ES1 energy is 0.5-1.0 ev larger than the ET1 energy thus resulting in low luminescence efficiency of pure organic fluorescent materials. Because of the unique molecular design, the thermal delayed fluorescence TADF materials can separate the HOMO-LUMO orbits and reduce the electron exchange energy of the two materials, so that ΔEST [symbol] 0 can be achieved theoretically. In order to effectively evaluate the thermal delayed fluorescence effect of the material in the invention, ΔEST evaluation is carried out.

The 1 wt % compound is doped into the mCBP film and the fluorescence and phosphorescence emission spectra are measured at 77K. By the relation of wavelength and energy, the value of S1 and T1 is converted to (E=1240/λem). Then, ΔEST=ES1−ET1 obtains the splitting energy of singlet and triplet. The data are shown in table 2:

TABLE 2

| Test item | SDF-DPSO2 | SDF-DPySO2 | SDF-DPSOcl2 | SDF-4PySOcl |
|---|---|---|---|---|
| ES1(ev) | 2.75 | 2.76 | 2.74 | 2.81 |
| ET1(ev) | 2.61 | 2.64 | 2.63 | 2.72 |
| ΔEST(ev) | 0.14 | 0.12 | 0.11 | 0.09 |

It can be seen from Table 2 that each compound of the present invention has a relatively small ΔEST value, which is less than 0.2 ev. Therefore, all of the compounds have the effect of thermal delayed fluorescence.

The invention also relates to a luminescent device, which is an organic light-emitting diode (OLED). It comprises an anode, a cathode and at least an organic layer arranged between the anode and the cathode, and the organic layer comprises an aromatic compound of the invention. Refer to FIG. 1 for a structural diagram of the luminescent device provided for the present invention. The luminescent device 10 includes the anode 11 formed in turn, a hole transport layer 12, a luminescent layer 13, an electron transport layer 14 and the cathode 15. Of which, thole transport layer 12, the luminous layer 13 and the electron transport layer 14 are all organic layers, and the anode 11 is electrically connected with the cathode 15.

The ITO substrate is a 30 mm×30 mm bottom emitting glass with four luminescent regions, covering a luminescent area of 2 mm×2 mm, and a transmittance of ITO thin film is 90%@550 nm, and its surface roughness Ra<1 nm, and its thickness is 1300 A, with square resistance of 10 ohms per square meters.

The cleaning method of ITO substrate as follows: first it is placed in a container filled with acetone solution, and the container is placed in ultrasonic cleaning machine for 30 minutes, in order to dissolve and remove most of the organic matter attached to the surface of ITO; and then the cleaned ITO substrate is removed and placed on the hot plate for half an hour at high temperature of 120 [symbol] , in order to remove most of the organic solvent and water vapor from the surface of the ITO substrate; and then the baked ITO substrate is transferred to the UV-ZONE equipment for processing with O³ Plasma, and the organic matter or foreign body which could not be removed on the ITO surface is further processed by plasma, and the processing time is 15 minutes, and the finished ITO is quickly transferred to the film forming chamber of the OLED evaporation equipment.

OLED preparation before evaporation: first of all, the OLED evaporation equipment is prepared, and then IPA is used to wipe the inner wall of the chamber, in order to ensure that the whole film chamber is free of foreign bodies or dust. Then, the crucible containing OLED organic material and the crucible containing aluminum particles are placed on the position of organic evaporation source and inorganic evaporation source in turn. By closing the cavity and taking the initial vacuum and high vacuum, the internal evaporation degree of OLED evaporation equipment can reach 10⁻⁷ Torr.

OLED evaporation film: the OLED organic evaporation source is opened to preheat the OLED organic material at 100 [symbol] for 15 minutes to ensure the further removal of water vapor from the OLED organic material. Then the organic material that needs to be evaporated is heated rapidly and the baffle over the evaporation source is opened until the evaporation source of the material runs out and the wafer detector detects the evaporation rate, and then the temperature rises slowly, the temperature rise is 1˜5° C., until the evaporation rate is stable at 1 A/s, the baffle directly below the mask plate is opened and the OLED film is formed. When it is observed that the organic film on the ITO substrate reaches the preset film thickness at the computer end, the mask baffle and the evaporative source directly above the baffle are closed, and the evaporative source heater of the organic material is closed. The evaporation process for other organic and cathode metal materials is described above.

OLED encapsulation process: the cleaning and processing of 20 mm×20 mm encapsulation cover is as the same as the pretreatment of ITO substrate. The UV adhesive coating or dispensing is carried out around the epitaxial of the cleaned encapsulation cover, and then the encapsulation cover of the finished UV adhesive is transferred to the vacuum bonding device, and stuck with the ITO substrate of the OLED film in vacuum, and then transferred to the UV curing cavity for UV-light curing at wavelength of 365 nm. The light-cured ITO devices also need to undergo post-heat treatment at 80 [symbol] for half an hour, so that the UV adhesive material can be cured completely.

(1) Performance Evaluation of Guest Luminescent Materials

In order to evaluate the electroluminescent properties of SDF-DPSO2 as guest luminescent material, OLED device structure ITO/NPB (30 nm)/TCTA (30 nm)/PPF: SDF-DPSO2 (x wt %, 30 nm, x=1-20)/PPF (10 nm)/TPBi (30 nm)/LiF (0.8 nm)/Al (150 nm) is designed.

The encapsulated sample is tested for IVL performance and IVL equipment is tested using Mc Science M6100, as shown in Table 3:

TABLE 3

| | Doping | Maximum external | |
|---|---|---|---|
| Device | ratio x | quantum efficiency | Maximum current |
| number | (wt %) | EQE (%) | efficiency (cd/A) |
| A | 1 | 9.8 | 26.5 |
| B | 5 | 11.0 | 29.7 |
| C | 10 | 14.9 | 38.2 |
| D | 15 | 14.1 | 36.5 |
| E | 20 | 13.3 | 35.3 |
| G(Firpic) | 10 | 20 | 54.1 |
| F(Firpic) | 10 | 15.7 | 40.9 |

The classic phosphor blue Firpic are used for comparing performance (No. F). The OLED device structure ITO/NPB (30 nm)/TCTA (30 nm)/PPF:SDF-DPSO2 (10 wt %, 30 nm)/PPF (10 nm)/TPBi (30 nm)/LiF (0.8 nm)/Al (150 nm) is designed. It can be found that the device performance based on SDF-DPSO2 increases with the increase of doping ratio, and the device performance tends to decrease after the doping ratio continues to increase, and the properties of SDF-DPSO2 with doping ratio of 10 wt % are very close to those of phosphor blue.

(2) Evaluation of Photoelectric Performance of the Host Material:

The device fabrication process is described above.

OLED device structure (number G) and OLED device structure ITO/NPB/TCTA/SDF-DPSO2: Firpic (10 wt %/SDF-DPSO2 (10 nm)/TPBI (30 nm)/LiF (0.8 nm)/Al are deisgned. It is found that the performance of G device is much better than that of F, that's because SDF-PSO materials have the ability to transfer both electrons and holes at the same time. Moreover, the HOMO-LUMO of SDF-DPSO2 is larger than that of HOMO-LUMO of Firpic, so its energy transfer is good.

Although the application is disclosed as above in a better embodiment, it is not used to define the claim, and any skilled person in the field may make a number of possible changes and modifications without departing from the concept of the application. The scope of protection of this application shall therefore be governed by the scope defined in the claim.

It is to be understood, however, that even though numerous characteristics and advantages of the present exemplary embodiments have been set forth in the foregoing description, together with details of the structures and functions of the embodiments, the disclosure is illustrative only, and changes may be made in detail, especially in matters of shape, size, and arrangement of parts within the principles of the invention to the full extent indicated by the broad general meaning of the terms where the appended claims are expressed.

What is claimed is:

1. A spirobifluorene compound being selected from the compound shown by the general formula I: 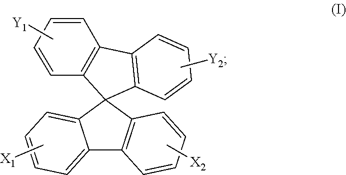
where, Y1, Y2 denote hydrogen, electron-absorbing groups or electron-donating groups independently, respectively, and at least one of the substituents in X1 and X2 is the substituent shown in formula II: 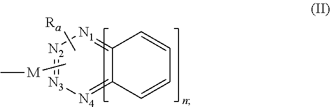
M denotes —S—, —P—, —SO—, —SO₂—, —S(═S)—, —S(═S)(═S)—, —PO—, —PO₂—, —P(═S)—, —P(═S)(═S)—, —C(═O)—;
N₁, N₂, N₃ and N₄ denote carbon or nitrogen atoms independently, respectively;
Rₐ is selected from hydrogen, halogen, C_{1˜30} alkyl, C_{1˜30} alkyl substituted by hydroxyl or C_{6˜48} alkylaryl;
N is an integer of 0˜4.

2. The spirobifluorene compound as described in claim 1 further being selected from the compound shown in the general formula IA:

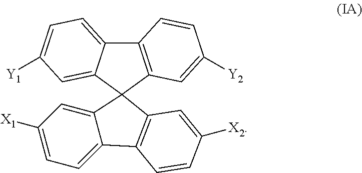

3. The spirobifluorene compound as described in claim 1, wherein the electron-donating groups are selected from the substituted or unsubstituted C_{1˜30} alkyl groups, the substituted or unsubstituted diphenyl groups, or the substituents denoted by the following structural expressions: 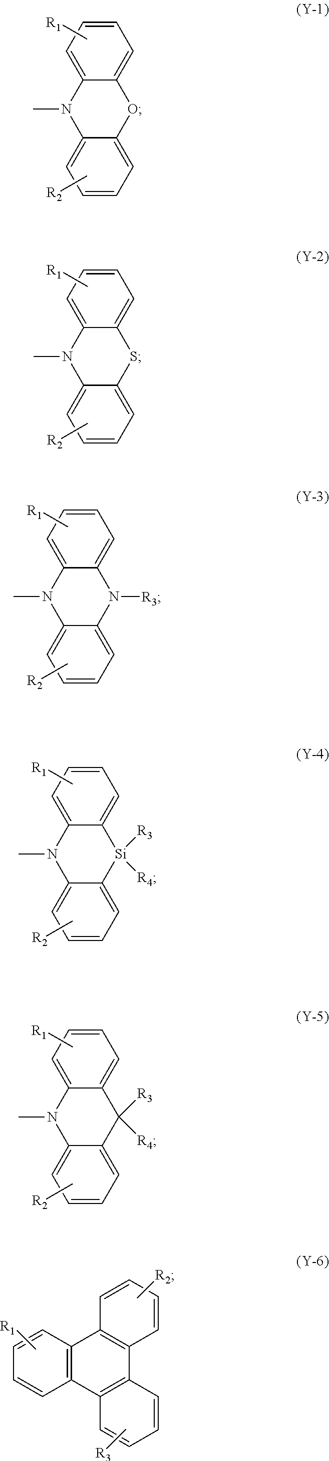 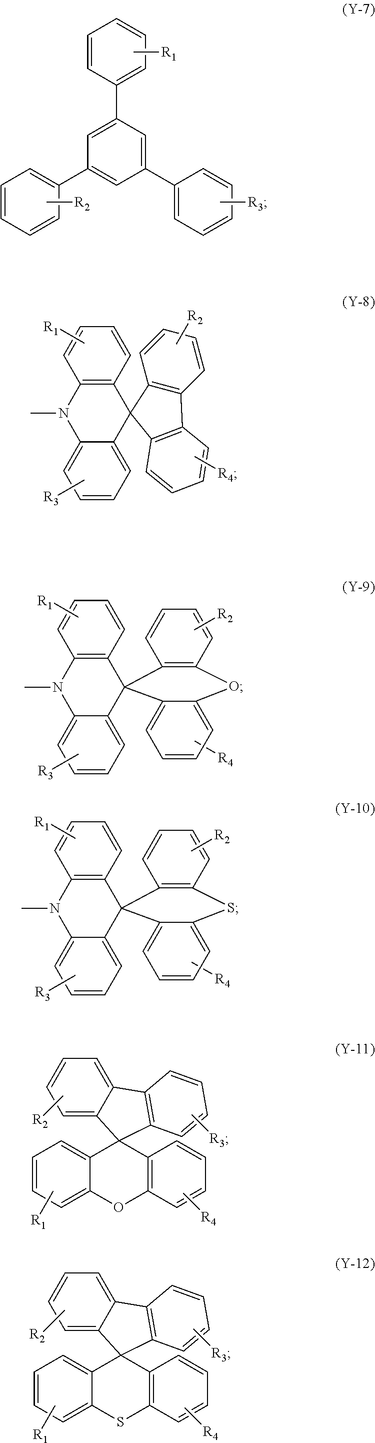 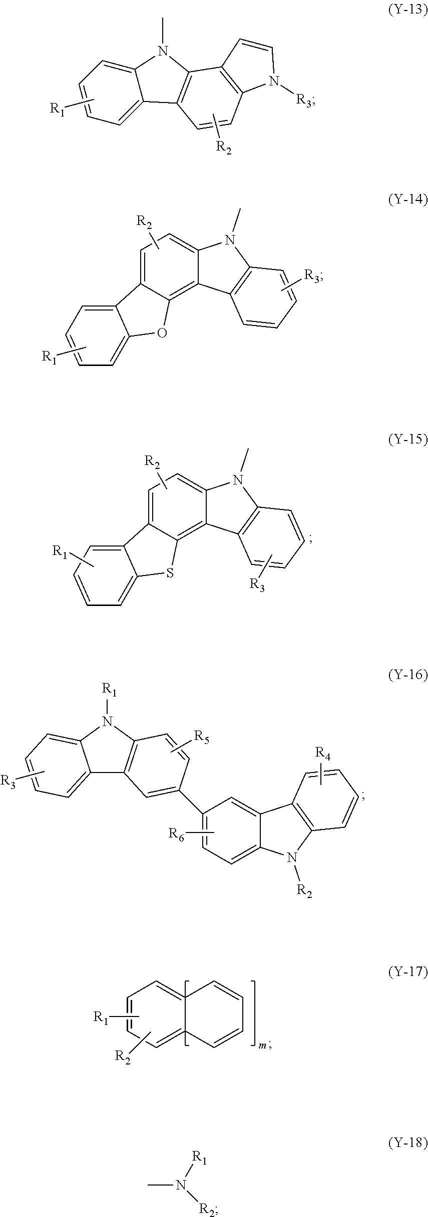
where, R₁, R₂, R₃, R₄ are selected from hydrogen atoms, amino groups, halogens, substituted or unsubstituted C_{1˜12} alkyl, substituted or unsubstituted C_{1˜12} alkoxyl, substituted or unsubstituted C_{6˜12} aryl groups, substituted or unsubstituted C_{6˜12} aryloxy groups;
the substituents are halogen atoms, alkyl groups of C_{1˜12}, alkyl groups of C_{1˜12} substituted by halogen atoms and alkoxyl groups of C_{1˜12} substituted by halogen atoms;
m is an integer of 0˜4;
any hydrogen atom on the benzene ring in the groups shown in the formula Y-6, the formula Y-7, the formula Y-11, the formula Y-12, the formula Y-16 and the formula Y-17 may be substituted to form a substituent.

4. The spirobifluorene compound as described in claim 1, wherein the electron-absorbing group is selected from the substituents shown in formula II.

5. The spirobifluorene compound as described in claim 1, wherein the X₁ is selected from the hydrogens in formula IIa, and X₂ is selected from the substituents shown in the formula IIa; 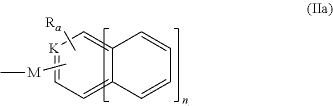
where, K denotes a carbon or a nitrogen atom.

6. The spirobifluorene compound as described in claim 1, wherein the spirobifluorene compound is selected from the compounds shown in the general formula IA-1: 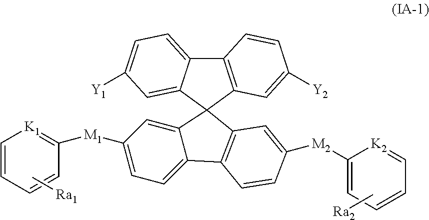
in IA-1,
M₁, M₂ denote —SO—, —SO₂—, —PO-independently, respectively;
Rₐ₁, Rₐ₂ are selected from hydrogen, halogen, C_{1˜12} alkyl, C_{6˜24} aryl groups independently, respectively;
K₁, K₂ denote carbon or nitrogen atoms independently, respectively.

7. The spirobifluorene compound as described in claim 1, wherein the spirobifluorene compound is selected from the compounds shown in the general formula IA-2: 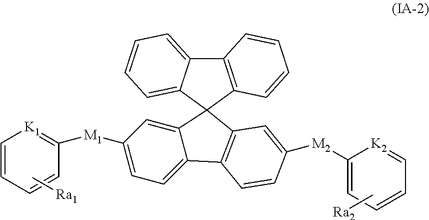
in IA-2,
M₁, M₂ denote —SO—, —SO₂—, —PO-independently, respectively;
Rₐ₁, Rₐ₂ are selected from hydrogen, halogen, C_{1˜12}alkyl, C_{6˜24} aryl groups independently, respectively;
K₁, K₂ denote carbon or nitrogen atoms independently, respectively.

8. The spirobifluorene compound as described in claim 1 wherein the spirobifluorene compound is selected from the compounds shown in the general formula IA-3: 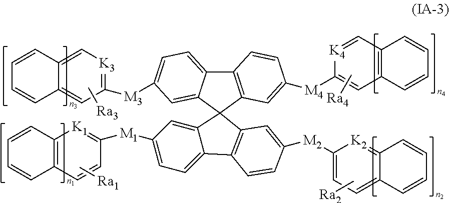
in IA-3, M₁, M₂, M₃, M₄ denote —SO—, —SO₂—, —PO-independently, respectively;
Rₐ₁, Rₐ₂, Rₐ₃, Rₐ₄ are selected from hydrogen, halogen, C_{1˜12} alkyl groups and C_{6˜24} aryl groups independently, respectively;
K₁, K₂, K₃, K₄ denote carbon or nitrogen atoms independently, respectively;
n₁, n₂, n₃, n₄ are selected from any integer of 0˜4.

9. The spirobifluorene compound as described in claim 7, wherein Rₐ₁, Rₐ₂, Rₐ₃, Rₐ₄ are selected from hydrogen atoms or halogens independently, respectively.

10. The spirobifluorene compound as described in claim 2 wherein the spirobifluorene compound is selected from a compound shown by the following structural formulas:

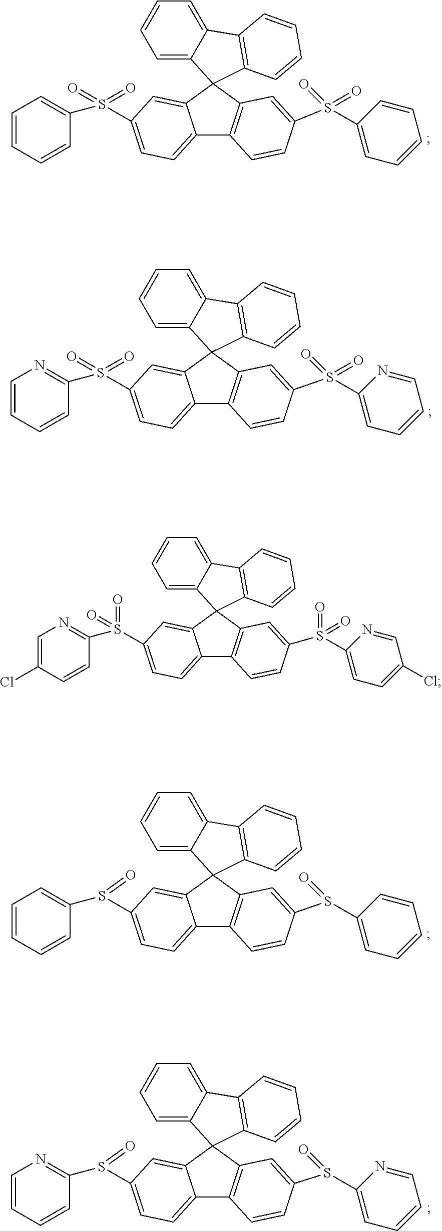 [SMILES: O=S(=O)(c1ccccc1)c1ccc2c(c1)C1(c3ccccc3-c3ccccc31)c1cc(S(=O)(=O)c3ccccc3)ccc1-2 ; O=S(=O)(c1ccc2c(c1)C1(c3ccccc3-c3ccccc31)c1cc(S(=O)(=O)c3ccccn3)ccc1-2)c1ccccn1 ; O=S(=O)(c1ccc2c(c1)C1(c3ccccc3-c3ccccc31)c1cc(S(=O)(=O)c3ccc(Cl)cn3)ccc1-2)c1ccc(Cl)cn1 ; O=S(c1ccccc1)c1ccc2c(c1)C1(c3ccccc3-c3ccccc31)c1cc(S(=O)c3ccccc3)ccc1-2 ; O=S(c1ccc2c(c1)C1(c3ccccc3-c3ccccc31)c1cc(S(=O)c3ccccn3)ccc1-2)c1ccccn1]

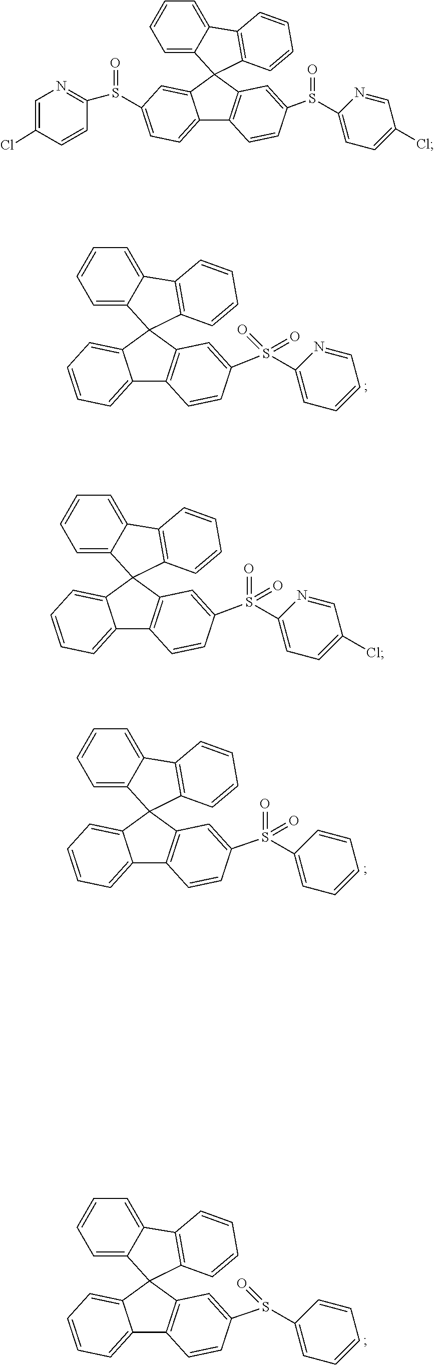 [SMILES: O=S(c1ccc2c(c1)C1(c3ccccc3-c3ccccc31)c1cc(S(=O)c3ccc(Cl)cn3)ccc1-2)c1ccc(Cl)cn1 ; O=S(=O)(c1ccc2c(c1)C1(c3ccccc3-c3ccccc31)c1ccccc1-2)c1ccccn1 ; O=S(=O)(c1ccc2c(c1)C1(c3ccccc3-c3ccccc31)c1ccccc1-2)c1ccc(Cl)cn1 ; O=S(=O)(c1ccccc1)c1ccc2c(c1)C1(c3ccccc3-c3ccccc31)c1ccccc1-2 ; O=S(c1ccccc1)c1ccc2c(c1)C1(c3ccccc3-c3ccccc31)c1ccccc1-2]

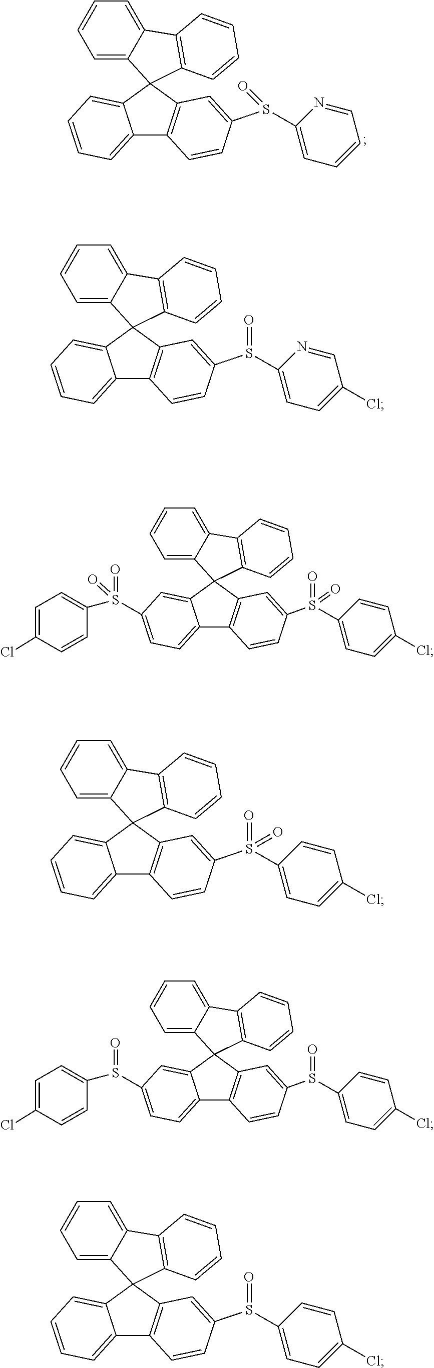 [SMILES: O=S(c1ccc2c(c1)C1(c3ccccc3-c3ccccc31)c1ccccc1-2)c1ccccn1 ; O=S(c1ccc2c(c1)C1(c3ccccc3-c3ccccc31)c1ccccc1-2)c1ccc(Cl)cn1 ; O=S(=O)(c1ccc(Cl)cc1)c1ccc2c(c1)C1(c3ccccc3-c3ccccc31)c1cc(S(=O)(=O)c3ccc(Cl)cc3)ccc1-2 ; O=S(=O)(c1ccc(Cl)cc1)c1ccc2c(c1)C1(c3ccccc3-c3ccccc31)c1ccccc1-2 ; O=S(c1ccc(Cl)cc1)c1ccc2c(c1)C1(c3ccccc3-c3ccccc31)c1cc(S(=O)c3ccc(Cl)cc3)ccc1-2 ; O=S(c1ccc(Cl)cc1)c1ccc2c(c1)C1(c3ccccc3-c3ccccc31)c1ccccc1-2]

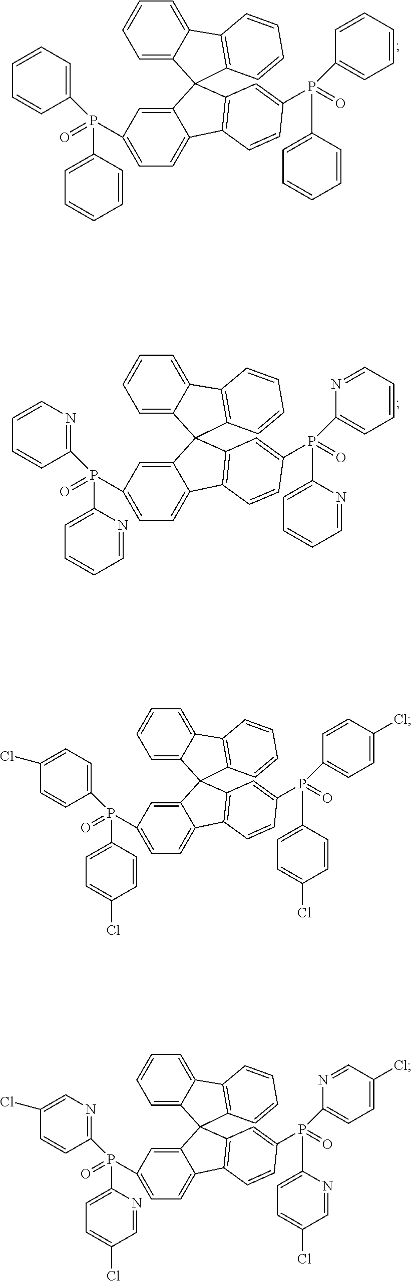 [SMILES: O=P(c1ccccc1)(c1ccccc1)c1ccc2c(c1)C1(c3ccccc3-c3ccccc31)c1cc(P(=O)(c3ccccc3)c3ccccc3)ccc1-2 ; O=P(c1ccc2c(c1)C1(c3ccccc3-c3ccccc31)c1cc(P(=O)(c3ccccn3)c3ccccn3)ccc1-2)(c1ccccn1)c1ccccn1 ; O=P(c1ccc(Cl)cc1)(c1ccc(Cl)cc1)c1ccc2c(c1)C1(c3ccccc3-c3ccccc31)c1cc(P(=O)(c3ccc(Cl)cc3)c3ccc(Cl)cc3)ccc1-2 ; O=P(c1ccc2c(c1)C1(c3ccccc3-c3ccccc31)c1cc(P(=O)(c3ccc(Cl)cn3)c3ccc(Cl)cn3)ccc1-2)(c1ccc(Cl)cn1)c1ccc(Cl)cn1]

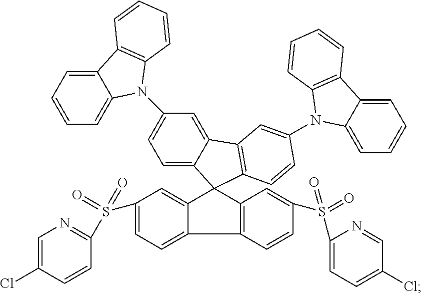 [SMILES: O=S(=O)(c1ccc2c(c1)C1(c3ccc(-n4c5ccccc5c5ccccc54)cc3-c3cc(-n4c5ccccc5c5ccccc54)ccc31)c1cc(S(=O)(=O)c3ccc(Cl)cn3)ccc1-2)c1ccc(Cl)cn1]

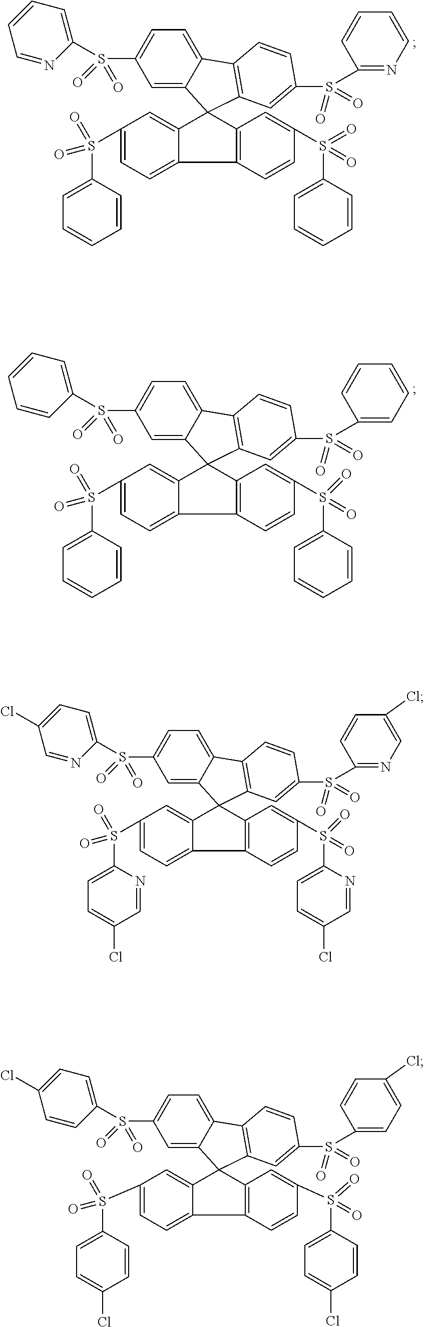

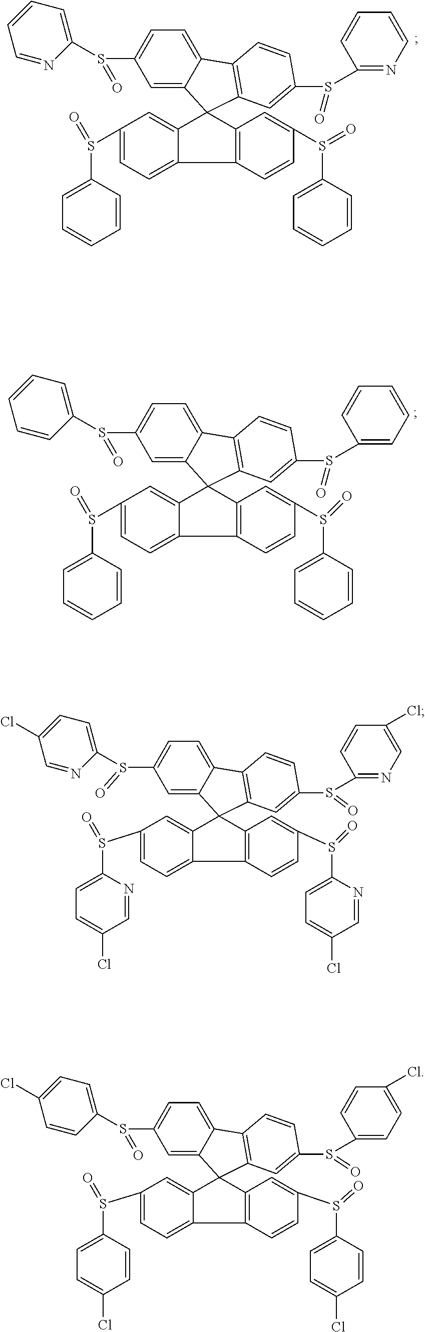 [SMILES: O=S(c1ccccc1)c1ccc2c(c1)C1(c3cc(S(=O)c4ccccc4)ccc3-2)c2cc(S(=O)c3ccccn3)ccc2-c2ccc(S(=O)c3ccccn3)cc21 ; O=S(c1ccccc1)c1ccc2c(c1)C1(c3cc(S(=O)c4ccccc4)ccc3-2)c2cc(S(=O)c3ccccc3)ccc2-c2ccc(S(=O)c3ccccc3)cc21 ; O=S(c1ccc2c(c1)C1(c3cc(S(=O)c4ccc(Cl)cn4)ccc3-2)c2cc(S(=O)c3ccc(Cl)cn3)ccc2-c2ccc(S(=O)c3ccc(Cl)cn3)cc21)c1ccc(Cl)cn1 ; O=S(c1ccc(Cl)cc1)c1ccc2c(c1)C1(c3cc(S(=O)c4ccc(Cl)cc4)ccc3-2)c2cc(S(=O)c3ccc(Cl)cc3)ccc2-c2ccc(S(=O)c3ccc(Cl)cc3)cc21]

11. A luminescent device comprising an anode, a cathode and at least an organic layer arranged between the anode and the cathode, wherein the organic layer comprises a spirobifluorene compound as described in claim 1.
